(19) United States
(12) Patent Application Publication
(10) Pub. No.: US 2023/0157550 A1
(43) Pub. Date: May 25, 2023

(54) CONTACTLESS VITAL SIGN MONITORING OF MULTIPLE SUBJECTS IN REAL-TIME

(71) Applicant: Kratos SRE, Inc., San Diego, CA (US)

(72) Inventors: Seth Daniel Cohan, Mountain Brook, AL (US); Stacey Suzanne Kelpke, Birmingham, AL (US); Jacob Matthew Robinson, Homewood, AL (US)

(21) Appl. No.: 17/917,487

(22) PCT Filed: Apr. 6, 2021
(86) PCT No.: PCT/US2021/025892

§ 371 Date: Oct. 6, 2022

Related U.S. Application Data
(60) Provisional application No. 63/005,807, filed on Apr. 6, 2020.

(51) Int. Cl. A61B 5/00; A61B 5/01; A61B 5/1171; G06V 40/16; G06V 40/10

(52) U.S. Cl. CPC A61B 5/0077; A61B 5/015; A61B 5/1176; G06V 40/168; G06V 40/15

(57) ABSTRACT

Systems and methods are provided to monitor vital signs from a plurality of subjects simultaneously and in real-time. The systems and methods can utilize visible video signal or infrared (IR) video signals, or both, defining respective sequences of frames representative of a scene including the plurality of subjects. The systems and methods can analyze digital images corresponding to respective frames in a sequence of frames to generate a time series of image values. In an aspect, the systems and methods can analyze the time series to identify a characteristic frequency representing a value of a vital signal that is oscillatory in nature.

### CROSS-REFERENCE TO RELATED APPLICATIONS

This application claims the benefit of U.S. Application No. 63/005,807, filed on Apr. 6, 2020, the contents of which are hereby incorporated by reference in their entirety.

### BACKGROUND

Some technologies for real-time physiological status monitoring (RT-PSM) can assess an individual subject by means of wearable devices, such as personal status monitors. Those technologies can report values of vital signs using the wearable devices. Other technologies can generate values of vital sign in a contactless fashion, without reliance on wearable devices. To that end, those contactless technologies can record video of a subject in data storage, and can then determine values of a vital sign by analyzing the video data a posteriori—that is, by processing the recorded video offline, after being recorded. The values of the vital sign can then be reported using an active technique, such as a radar.

Further, contactless technologies that rely on visible video and infrared (IR) video for vital signs monitoring can be subject to various technical challenges arising from environmental conditions, such as, ambient lighting, humidity level, floor reflectivity, thermal resolution, camera field of view (FOV), and so forth. Not only can those technical challenges influence IR sensitivity and/or tracking algorithm reliability, but they also can limit the environments and distances over which an RT-PSM can operate.

Therefore, much remains to be improved in technologies for real-time monitoring of vital signs. Improved technologies that address the issues mentioned above, amongst others, may be desirable.

### SUMMARY

Commonplace technologies for real-time physiological status monitoring (RT-PSM) technologies are aimed at providing information on a patient's vitals. Embodiments of the technologies disclosed herein can use video camera streams in the visible and infrared to monitor the vitals from one or more subjects simultaneously and in real-time. The vitals include heart rate, respiratory rate, temperature, and the like. The subjects being monitored can include those that can be seen in live output of the video camera streams.

In an aspect, in contrast with conventional technologies for monitoring of vital signs, disclosed herein systems and methods can be reported in real-time using only video data streams. In further contrast, the systems and methods can simultaneously report values of vital signs from multiple subjects in the same video data streams.

In an aspect, the systems and methods can enhance and augment commonplace vital monitoring devices that require a wearable device on subject (e.g., a hospital patient, an athlete, a passenger in a transportation system, or similar). The disclosed systems and methods also can provide health information in situations where wearable devices may not ideal and cannot operate efficiently. Those situations can include non-contact scenarios, such as neonatal infants or patients with hyper-sensitivity to physical stimulation; large-scale areas where vitals monitoring of crowds is desired or otherwise required; situations that require vitals monitoring by individuals with limited or no training beyond camera operation; and others. Because the systems and methods can permit monitoring multiple subjects simultaneously, the cost of deploying vitals monitoring capabilities can be reduced, more particularly in remote locations with lesser developed infrastructures, such as, for example in medical environments.

In another aspect, in sharp contrast to conventional technologies, the systems and methods can provide video-based vitals monitoring that can increase (or even maximize) the number of vital signs and the number of subjects that can be monitored simultaneously in nearly real-time. Further, the systems and methods also can decrease the amount of equipment needed to monitor the vital signs of multiple subjects and does so in a passive, contactless manner. By monitoring vital signs without wearable devices, additional care can be provided to subjects with skin sensitivity and other impairments. Contactless vital signs monitoring disclosed herein can be used for fatigue monitoring of individuals without interrupting normal activities, for example. Also, the systems and methods can be applied in high-stress working environments without the added stress of attached sensors and/or other wearable devices.

In an aspect, the systems and methods also can include machine-learning (ML) techniques that can enable automated recognition of subtle or complex manifestations of illness/injury. As such, embodiments that include ML techniques can generate data that can facilitate implementations of ML diagnostic tools.

Additional features or advantages of the disclosed systems and methods will be set forth in part in the description which follows, and in part will be apparent from the description, or may be learned by practice of this disclosure. The advantages of the disclosure can be realized and attained by means of the elements and combinations particularly pointed out in the appended claims. It is to be understood that both the foregoing general description and the following detailed description are exemplary and explanatory only and are not restrictive of the subject disclosure.

### BRIEF DESCRIPTION OF THE DRAWINGS

The annexed drawings are an integral part of the disclosure and are incorporated into the present specification. The drawings illustrate examples of embodiments of the disclosure and, in conjunction with the description and claims, serve to explain, at least in part, various principles, features, or aspects of the disclosure. Some embodiments of the disclosure are described more fully below with reference to the drawings. However, various aspects and elements of the disclosure can be implemented in many different forms and should not be construed as being limited to the implementations set forth herein. Like numbers refer to like, but not necessarily the same or identical, elements throughout. The accompanying drawings can be briefly characterized as follows.

FIG. 1 is an exemplary vital-sign monitoring environment.

FIG. 2 is an exemplary vital-sign monitoring system.

FIG. 3 is an exemplary graphical user interfaces (GUIs) provided in a vital-sign monitoring environment.

FIG. 4 is another exemplary vital-sign monitoring environment.

FIG. 5 is an exemplary a vita-sign monitoring system.

FIG. 6 illustrates an example of another vital-sign monitoring environment.

FIG. 7 is a process flowchart of an exemplary method.

FIG. 8 is a process flowchart of another exemplary method.

FIG. 9 is an exemplary computing environment that can be utilized for monitoring vitals.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

In one aspect of the disclosure, methods and systems can recognize and address, amongst other technical challenges, the issue of contactless monitoring vital signs in real-time. In an aspect, the methods and systems can monitor vital signs from multiple individuals simultaneously and in real-time. This is possible by using optimized software for streaming high-resolution frames of the video feed(s) directly into a computing system that can analyze important regions of each frame to compute metrics quickly and then move to a next frame. The frame-by-frame analysis can be performed without persisting video data in a file.

Although embodiments of the disclosed have been described in connection with particular vital signs, such as skin temperature, body temperature, heart rate, and respiration rate, the methods and systems are not limited in that respect. Indeed, the methods and systems can monitor other vital signs of multiple subjects in nearly real-time. Simply for purposes of illustration, those other vital signs can include, blood pressure; circulation; oxygen saturation (SPO₂); or similar. For example, oxygen saturation levels can be estimated from visible video signals using a ratio of blue pixels and red pixels in digital images of subject skin. In addition, or some embodiments, other bodily functions or states can be monitored in nearly real-time. As an illustrations, the bodily functions or states can include sleep patterns; mouth vs. nose breathing; brain activity levels or physical activity levels; blink rates; estimated stress levels; identification of coughing or sneezing; eye movement; perspiration; facial expressions and/or mood; discomfort or pain from facial expressions; seizures; facial swelling; or similar. In some situations, the monitoring of one or many of such bodily functions can be accomplished by analyzing facial features and other qualitative visual cues. For example, the facial features and/or other quantitative visual cues can be quantified/classified through video analysis by pre-trained processes, where the training has occurred prior to the real-time monitoring of a bodily function or a bodily state, or both. Quantified outputs can include probabilities associated with the output classification(s).

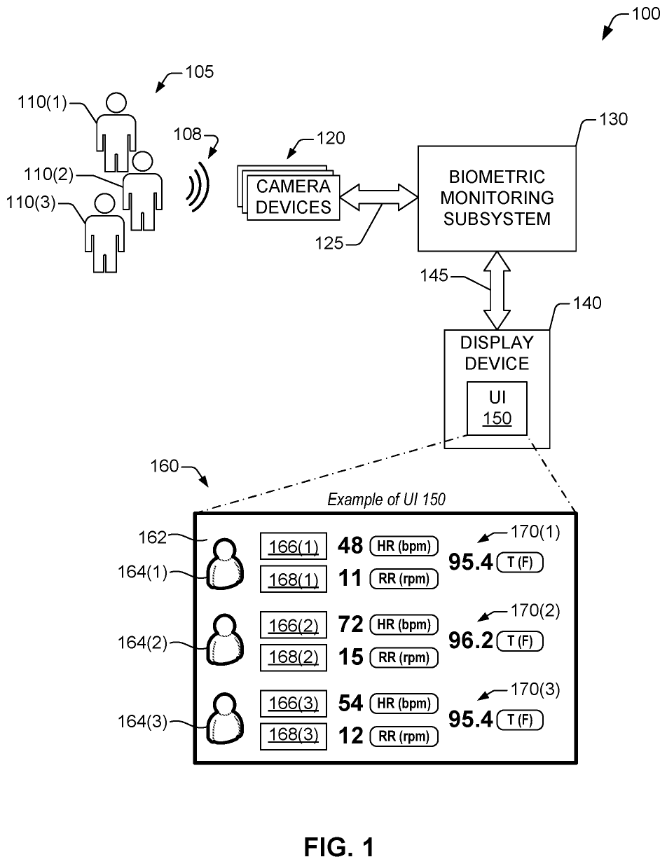

FIG. 1 is a schematic block diagram of an example of a vital-sign monitoring environment 100, in accordance with one or more embodiments. The monitoring environment 100 permits determining values of vital signs of multiple subjects simultaneously and in nearly real-time. The determination of the values of the vital signs can be performed nearly continuously. A time interval between successive determination of values of a vital sign can be dictated by processing conditions (processing load, for example) and/or other image acquisition rate involved in the determination of a value of a vital sign.

To that end, the monitoring environment 100 includes a plurality of camera devices 120 and a biometric monitoring subsystem 130. The plurality of camera devices 120 can generate video signals of a scene including multiple subjects. To that end, the plurality of cameras 120 are configured to detect light 108 in a particular portion of the electromagnetic radiation spectrum. In one configuration, the plurality of camera devices 120 can be distributed in space in order to generate video signals of the scene from different vantage points. In one of those configurations, at least one first camera can have respective first vantage point(s) and associated first field(s) of view, and at least one second camera can have respective second vantage point(s) and associated second field(s) of view.

The plurality of camera devices 120 can send the video signals to the biometric monitoring subsystem 130 as the video signal is generated. The biometric monitoring subsystem 130 can generate values of respective vital signs in nearly real-time, for the multiple subjects simultaneously. Those values are generated using the video signal as the video signal is received at the biometric monitoring subsystem 130.

In an aspect, the video signal can include visible video signal or infrared (IR) video signal, or a combination of both. As an example, the visible video signal can include video data defining a sequence of frames, where each frame can represent a digital image formed using visible light. An as example, the infrared video signal can include video data defining a sequence of frames, where each frame can represent a digital image formed using infrared light. For example, visible frames and infrared frames can be generated at respective acquisition rates (25 fps, for example). The acquisition rate also can be a variable frame rate. As such, at least one first camera device of the plurality of camera devices 120 can detect light in the visible portion of the electromagnetic (EM) radiation spectrum—e.g., each of the at least one first camera device can sense photons having energies in a range from about 1.63 eV to about 3.26 eV. In some scenarios, at least one of the camera devices 120 can leverage multiple wavelengths of the light in order to improve the signal-to-noise ratio and/or accuracy of vitals (e.g., red and blue filters). In addition, at least one second camera device of the plurality of camera devices 120 can detect light in the infrared portion of the EM radiation spectrum—e.g., each of the at least one second camera device can sense photons having energies in a range from about 1.24 meV to about 1.63 eV.

In one aspect, the plurality of camera devices 120 can send the visible video signal and the infrared video signal separately, in respective video channels. In other configurations, the plurality of camera devices 120 can multiplex the visible video signal and the infrared video signal into a single video channel. Regardless of the manner in which the video signals are supplied, the biometric monitoring subsystem 130 can receive the visible video signal or the infrared video signal, or both. The biometric monitoring subsystem 130 can then analyze the received video signal to identify facial features (e.g., eyes, nose, mouth, ears, forehead) or other body features (e.g., thorax, hand, arm, foot, leg, etc.) within the scene including the multiple subjects 105. Rather than analyzing the video signal after the acquisition of a video segment of the scene is completed, the biometric monitoring subsystem 130 can analyze the video signal as the video signal is received (e.g., in real-time).

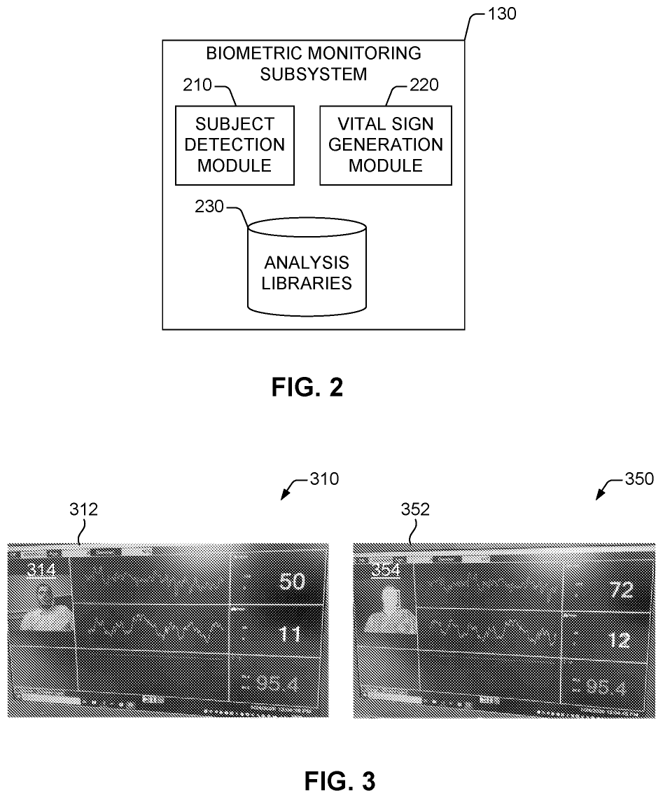

In an aspect, the biometric monitoring subsystem 130 can apply a feature tracking technique to detect a group of facial features in each frame of the sequence of frames included in the received video signal. The facial features can include, in one configuration, faces, noses, foreheads, or a combination of those features, for example. In a further aspect, as is illustrated in FIG. 2, the biometric monitoring subsystem 130 can include a subject detection module 210 that can apply the feature tracking technique. The biometric monitoring subsystem 130 also can include one or more analysis libraries 230 that include data defining the feature tracking technique. The analysis libraries 230 can be memory devices.

The biometric monitoring subsystem 130 can assign subgroups of the group of facial features to respective subjects present in respective scenes represented by the sequence of frames. More specifically, as an illustration, the biometric monitoring subsystem 130 can detect a first group of facial features in a frame in the sequence of frames. The biometric monitoring subsystem 130 can then assign a first subgroup of the first group of facial features with the first subject 110(1), a second subgroup of the first group of facial features to the second subject 110(2), and a third subgroup of the group of facial features to the third subject 110(3). In a consecutive (either successive or subsequent) frame in the sequence of frames, the biometric monitoring subsystem 130 can detect a second group of facial features. The biometric monitoring subsystem 130 can then assign a first subgroup of the second group of facial features with the first subject 110(1), a second subgroup of the second group of facial features to the second subject 110(2), and a third subgroup of the second group of facial features to the third subject 110(3). The biometric monitoring subsystem 130 can further assign a fourth subgroup of the group of facial features to another subject that has entered the scene represented by the successive frame.

In an aspect, the biometric monitoring subsystem 130 can track at least some of the multiple subjects present in a scene. A configurable parameter can define a threshold number of subjects to be tracked. The biometric monitoring subsystem 130 can use the defined threshold to limit a maximum or a minimum number of subjects being tracked during a particular time interval.

In an aspect, the biometric monitoring subsystem 130 can assign a group of facial features to a subject in several ways. In some example cases, the group of facial features can be assigned to the subject anonymously, where the biometric monitoring subsystem 130 generates an association between the group and the subject, without including data that identify or can be used to identify the subject.

In other example cases, the group of facial features can be assigned to the subject and a record that identifies the subject can be created. In those cases, the biometric monitoring subsystem 130 generates an association between the group and the subject. The biometric monitoring subsystem 130 can then obtain identification data pertaining to the subject from a data repository (not depicted in FIG. 1). For that purpose, the biometric monitoring subsystem 130 can query a database including a preset record of faces. The database can be retained in the repository. As an example, a record of faces can be created when a patient enters into the care of a hospital, by photographing the patient and retaining the photograph (e.g., a headshot) in the database. As a result, the biometric monitoring subsystem 130 can recognize the face of the subject a first time the subject is within the field of view (FOV) of one or many of the multiple camera devices 120. The biometric monitoring subsystem 130 can use the identification data (e.g., electronic medical record ID) to retain values of a vital sign of the subject within an electronic health record of the subject. In some embodiments, the subject detection module 210 (FIG. 2) can assign a group of facial features to a subject, either anonymously or identifiably (i.e., recorded identification data).

By assigning a subgroup of facial features to a subject in a digital image corresponding to a frame, the biometric monitoring subsystem 130 can determine multiple regions of interest within the digital image. Thus, for multiple subjects in the digital image (e.g., first subject 110(1), second subject 110(2), and third subject 110(3)) the biometric monitoring subsystem 130 can determine respective regions of interest.

In an aspect, the biometric monitoring subsystem 130 can determine values of vital signs by analyzing the multiple regions of interest. To that point, each one of the multiple regions of interest within a frame k includes an array of pixels. Here, k is a frame index. The biometric monitoring subsystem 130 can operate on image data contained in the array of pixels to determine an image value for the region of interest (ROI). In an aspect, the biometric monitoring subsystem 130 can determine the image value by evaluating a defined function ƒ(·) that is single-valued and represents the image content of pixels contained in ROI. Without intending to be bound by theory and/or modeling, the defined function ƒ(·) can be the average pixel values (e.g., color values or brightness values) over the pixels contained in the ROI. The average value that results from evaluating ƒ(·) is the image value corresponding to the ROI within the frame k.

Because the ROI corresponds to a particular subject a, the image value also corresponds to the particular subject a. Hence, by operating on the regions of interest present in the frame k, the biometric monitoring subsystem 130 can generate a set of image values {Iₖ^{σ}} for α=1, 2 . . . M-1, M, where M is the number of subjects detected in the frame k.

For a next frame k′ in the sequence of frames, the biometric monitoring subsystem 130 can repeat the detection of regions of interest within the digital image corresponding to the next frame k′. Again, those regions of interest correspond to respective subjects within the digital image, and include respective arrays of pixels. The vital-sign monitoring device 130 can generate another set of image values {Iₖ^{(σ′)}} for σ′=1, 2 . . . M′-1, M′, where M′ is the number of subjects detected in the frame k′. Although M′ can be equal to M for successive frames, M and M′ can be different in some situations.

By repeating the detection of facial features and assigning regions of interest across a sequence of frames, the biometric monitoring subsystem 130 can determine respective sets of image values {Iₖ^{(σ)}} for a defined group of subjects σ=1, 2, . . . N, N-1, where k=1, 2 . . . n-1, n, with n a natural number defining a number of frames in the sequence of frames. The number n can be referred as a sampling number, and is a configurable parameter utilized for the determination of a value of a vital sign. As mentioned, the biometric monitoring subsystem 130 can receive video signal nearly continuously from the camera devices 120. Thus, the sampling number n defines a portion of the received frames and also can define an update rate for a value of a vital signal determined using the video signal.

For a particular subject Σ (e.g., subject 110(3)) the set {Iₖ^{(Σ)}}, with k=1, 2 . . . n-1, n, constitutes a time series that can be used to determine a value of a vital sign. To that point, for a vital sign corresponding to an oscillatory physiological quantity (e.g., the pumping of blood by a heart or the intake of air at a lung) a value of the vital sign can be obtained by transforming the time series from time domain to frequency domain. By updating the time series to include new frames received as the video signal is generated by the camera devices 120, the value of the vital sign can be updated at the rate in which the time series is updated. As a result, updated values of the vital sign can be generated nearly continuously during the reception of the video signal. A time interval between successive updates of values of a vital sign can be dictated by various factors involved in the determination of a value of a vital sign. Those factors can include processing conditions (processing load of the biometric monitoring subsystem, for example) and/or other image acquisition rate (e.g., the video frame rate) of one or more of the camera devices 120. The image acquisition rate can establish a lower bound to rate of the real-time monitoring.

The biometric monitoring subsystem 130 can transform the time series from time domain to frequency domain by applying a suitable transformation, e.g., a discrete Fourier transform, a discrete wavelet transform, a discrete cosine transform, a discrete sine transform, or similar. As a result, the biometric monitoring subsystem 130 can generate multiple frequencies (also referred to as frequency components) that characterize the oscillatory nature of the vital sign. The biometric monitoring subsystem 130 can then analyze the multiple frequencies to select a particular frequency that satisfies a magnitude criterion. In one example, the magnitude criterion can dictate that a select frequency must have the largest magnitude in a set of frequencies. Accordingly, the particular frequency that is selected can be the frequency having the largest magnitude amongst the multiple frequencies. In another example, the magnitude criterion can dictate that a select frequency must have the second largest magnitude in the set of frequencies. Accordingly, the particular frequency that is selected can be the frequency having the second largest magnitude amongst the multiple frequencies.

Regardless of the specific magnitude criterion that is applied to select the particular frequency, the biometric monitoring subsystem 130 can determine a value of the vital sign using the particular frequency. To that end, the biometric monitoring subsystem 130 can convert the characteristic frequency to such a value in suitable units, e.g., beats per minute (bpm) or respirations per minute (rpm).

In some embodiments, instead of selecting a single frequency, the biometric monitoring subsystem 130 can determine a characteristic frequency by averaging generated frequencies having respective magnitudes that exceed a defined threshold magnitude. The biometric monitoring subsystem 130 can determine a value of the vital sign using the characteristic frequency. Again, the biometric monitoring subsystem 130 can convert the characteristic frequency to such a value in suitable units (e.g., bpm or rpm). In addition, or in other embodiments, the biometric monitoring subsystem 130 can determine the characteristic frequency by selecting a particular spacing of harmonics. The characteristic frequency also can be determined by applying specific filters to the multiple frequencies determined by the transformation from time domain to frequency domain. The frequency with the largest magnitude after applying the specific filters can then be selected as the characteristic frequency. Further, or in yet other embodiments, the biometric monitoring subsystem 130 can determine the characteristic frequency by pairing the largest frequency component with a value measured in the time-domain. As an example, the biometric monitoring subsystem 130 can analyze the time between zero crossings of an oscillatory wave along with the locations of the harmonics.

Although some vital signs may not be associated with oscillatory physiological quantities, the systems and methods still can be applied to monitor those types of vital signs in real-time for multiple subjects simultaneously. In some embodiments, skin temperature or body temperature can be determined in nearly real-time using infrared video signal. In those embodiments, the biometric monitoring subsystem 130 can identify particular facial features in a group of facial features associated with a subject. The biometric monitoring subsystem 130 can then determine a region containing the particular facial features within a digital image corresponding to an image frame of a sequence of infrared image frames. The biometric monitoring subsystem 130 can determine an image value representative of an average pixel intensity within the region. In some cases, the pixels included in the determination of the image value are those pixels having an intensity (e.g., brightness value) that exceeds a threshold. The biometric monitoring subsystem 130 can map the image value to a temperature value of the skin temperature or the body temperature, or both.

In some configuration, to determine skin temperature or body temperature, the biometric monitoring subsystem 130 can identify a first facial feature (e.g., forehead) using a visible video signal and also can identify a second facial feature (e.g., nose) using in the infrared video signal. In one example, the biometric monitoring subsystem 130 can apply a feature tracking technique on the visible video signal to identify the first facial feature—e.g., first facial feature can be forehead and the biometric monitoring subsystem 130 can identify an upper portion of a tracked face as the first facial feature. In one embodiment, the feature tracking technique can be retained in the analysis libraries 230 (FIG. 2). The biometric monitoring subsystem 130 can then obtain a spatial relationship (relative position, relative orientation, etc.) between a visible camera and an IR camera included in the camera devices 120. The spatial relationship can be obtained, for example, by generating the spatial relationship using data defining the spatial arrangement of the camera devices 120, including the visible camera and the IR camera. A determination of the spatial relationship can rely on a calibration of the visible camera and the IR camera. The calibration can be automated in order to maintain the nearly real-time aspects of the monitoring described herein.

Such a spatial relationship permits geometrically transforming a view of a face from the vantage point in one of the camera devices 120 (e.g., visible camera) to another view from the vantage point of one of the camera devices 120 (e.g., IR camera). Thus, the biometric monitoring subsystem 130 can use the spatial relationship to assign pixels in the IR video signal to the face that corresponds to the tracked first facial feature in the visible video signal. The biometric monitoring system 130 can use such pixels to identify a model face in the IR video signal. The model face represents locations of facial features (e.g., nose and mouth locations) in the IR video signal that correspond to the tracked face in the visible video signal. Such facial features in the IR video signal can be utilized to determine a skin temperature or a body temperature, or both.

By spatially correlating facial features (e.g., a face and nose) tracked in visible video signal with a model of the face and nose in the IR video signal in order to determine bodily temperatures, the systems and methods can avoid the complexities associated with tracking facial features using IR image frames. Accordingly, the bodily temperatures can be monitored in nearly real-time without introducing processing latency that may distort the values of such temperatures.

In order to map image values to temperatures, an IR calibration source can be placed in the FOV of an IR camera includes in the camera devices 120 in order to maintain a constant thermal reference. Similarly, visible sources and/or IR oscillatory sources can be used to calibrate the frequency measurements of the video feeds.

Further, a calibration procedure can be implemented in order to align skin and body temperature of a subject to image intensity in a digital image generated using IR light. For example, a procedure can comprise manually measuring the body temperature of an individual using a thermometer while simultaneously measuring skin temperature with a camera. The difference(s) between these numbers can be used to compute body temperature from skin temperature.

Referring to FIG. 2, the biometric monitoring subsystem 130 can include a vital sign generation module 220 that can transform a time series from time domain to frequency domain by applying a suitable transformation to the time series. The vital sign generation module 220 also can select a frequency resulting from such a transformation, and can convert the frequency to suitable units in order to determine a value of a vital sign. A technique to apply the transformation can be retained in the analysis library 230. The magnitude criterion also can be retained in the analysis libraries 230. In some embodiments, techniques retained in the analysis libraries to determine values of vital signs can be tuned in nearly real-time by an end-user.

Although the determination of values of a vital sign in nearly real-time has been disclosed in connection with the tracking of facial features, the systems and methods are not limited in that respect. In some embodiments, the biometric monitoring subsystem 130 can track other part of a body to determine values of the vital sign. In addition, or in other embodiments, the biometric monitoring subsystem 130 can utilize data from multiple separate areas of the body (hands, feet, or thorax, for example) to determine values of the vital sign.

In some embodiments, the biometric monitoring subsystem 130 can use markers or other types of token on the body of a subject to assist with tracking of the subject. The markers or tokens also can be utilized to detect a subject that is exempt from having their vital signs monitored. For instance, markers can be utilized to distinguish patients and doctors or other types of healthcare providers who are exempt from having their vital signs monitored. An example of such markers can include adhesive films or inks that may only be visible when subjected to IR light. Another example of those markers can include a name tag, a sticker, or an embroidery patch having specific markings (e.g., name and rank of a military officer).

Facial and other tracking can occur in, or can be facilitated by, all camera devices 120 simultaneously or in a subset of the camera devices 120. In a scenario of tracking with a subset of the camera devices 120, information from this subset can be used to locate tracked facial features or faces in non-tracking cameras by the utilizing relative positions, relative orientations, and FOVs of an arrangement of the camera devices 120. Prior calibration of such an arrangement also can be utilized.

Further, for a vital sign defined by oscillatory physiological events, the systems and methods can analyze the motion of the body or clothing to determine values of the vital sign. For instance, if a patient is intubated, the biometric monitoring subsystem 130 system can track chest movements to determine respiratory rate.

In addition, or in some embodiments, the biometric monitoring subsystem 130 also can use a record of “do-not-track” faces. Data defining such faces and, optionally, other identification records associated with the faces can be retained in the data repository utilized by the biometric monitoring system 130 to track faces in an identifiable manner, as is described herein. In one of those embodiments, the data defining “do-not-track” faces can be embodied in, or can form a part of, a database of medical staff who has opted out or is otherwise not required to have their vital signs monitored. Thus, when a subject corresponding to a do-not-track face is visible on camera, the biometric monitoring subsystem 130 (via the subject detection 210, for example) can recognize the subject and can initiate (or can continue, in some cases) monitoring without computing vital signs for such a subject.

Referring back to FIG. 1, the biometric monitoring subsystem 130 can send values of respective vital signs to a display device 140 upon determining those values. The values can be sent by means of a communication architecture 145. In response, the display device 140 can present a user interface (UI) 150. The UI 150 can include multiple visual elements representative of monitored subjects and values of vital signs. An example of the UI 150 is illustrated in diagram 160 in FIG. 1. The exemplified UI 150 includes a pane 162 including a first visual element 164(1) representing a first subject (e.g., subject 110(1)); a second visual element 164(2) representing a second subject (e.g., subject 110(2)); and a third visual element 164(3) representing a third subject (e.g., subject 110(3)).

As is further shown in the diagram 160, the exemplified UI also includes second panes presenting time-dependent signals corresponding to particular vital signs for each subject. More specifically, the exemplified UI includes a pane 166(1) and a pane 168(1) corresponding, respectively, to time dependent-signals of first and second vital signs of the first subject. The exemplified UI also includes a pane 166(2) and a pane 168(2) corresponding, respectively, to time dependent-signals of the first and second vital signs of the second subject. The exemplified UI further includes a pane 166(3) and a pane 168(3) corresponding, respectively, to time dependent-signals of the first and second vital signs of the third subject. Simply as an illustration, as is shown in the diagram 160, the first vital sign can be heart rate (HR) and the second vital sign can be respiration rate (RR).

Further, the exemplified UI 150 also includes a first group of visual elements 170(1) corresponding to the first subject. The first group of visual elements 170(1) identify monitored vital signs (e.g., HR, RR, and skin temperature (T)) and applicable units, and respective values of the monitored vital signs. A second group of visual elements 170(2) corresponding to the second subject also can be included in the exemplified UI 150. The second group of visual elements 170(2) identify monitored vital signs (e.g., HR, RR, and skin temperature (T)) and applicable units, and respective values of the monitored vital signs. The exemplified UI 150 further includes a third group of visual elements 170(3) corresponding to the third subject. The third group of visual elements 170(3) identify monitored vital signs (e.g., HR, RR, and skin temperature (T)) and applicable units, and respective values of the monitored vital signs.

Other types and/or layouts of visual elements can be included in the UI 150. For example, a diagram 310 and a diagram 350 shown in FIG. 3 illustrate examples of a UI 150 in which monitored vitals for a single subject are shown. In the diagram 310, a visible image 314 of the subject is included in a pane 312 of the exemplified UI 150. In the diagram 350, an infrared image 354 of the subject is included in a pane 352 of the exemplified UI 150. In another example, the UI 150 can present a layout of UI elements that is similar to the format utilized by commonplace vital-sign monitoring stations (e.g., nurse's stations).

In embodiments in which a subject is tracked in an identifiable fashion, as described herein, a customized number of vital signs can be presented in the UI 150. Such vital signs can include vital sign(s) specific to an existing health condition (sleep apnea, asthma, diabetes, or COPD, for example). For other ones of the monitored subjects, a different set of vital signs can be presented within the UI 150.

The display device 140 can update the UI 150 upon receiving new values of the vital signs. Updating the UI 150 can include redrawing the UI 150 in order to update a prior-presented visual element with a current visual element indicative of a value of a vital sign, for example.

In some embodiments, the UI 150 can include display parameters (layout, indicia, etc.) can be tuned in real time by an end-user. In addition, or in other embodiments, the UI 150 can include a method for manually defining regions of interest.

Because the monitoring technologies disclosed herein can report values of vital signs in nearly real time, these technologies can be used to provide live input information to autonomous feedback systems or automation control systems that can respond to changes in observed vital signs without reliance on wearable devices.

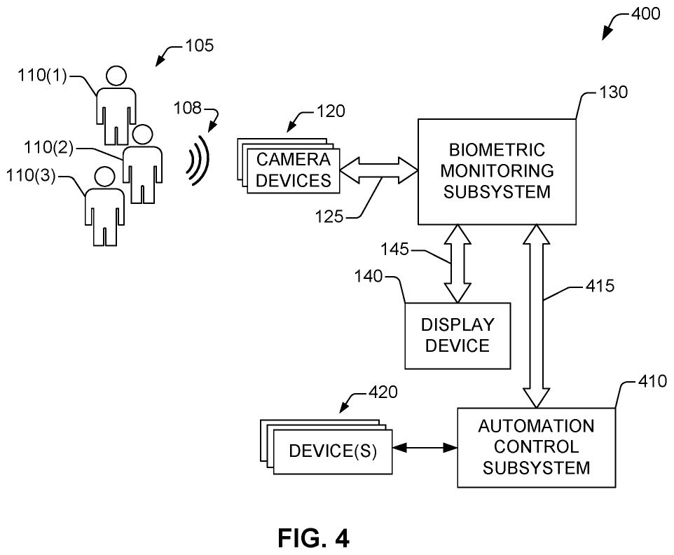

FIG. 4 illustrates an example of a vital sign monitoring environment 400 that can send instructions to an automation control subsystem 410 based at least on values of vital signs, in accordance with one or more embodiments. In a situation in which one or many values of vital signs, individually or collectively, satisfy a triggering criterion, the biometric monitoring subsystem 130 can cause the automation control subsystem 410 to direct one or more devices 420 to perform a defined operation. Such an operation can based at least on the value(s) of the vital signs. In some embodiments, the biometric monitoring subsystem 130 can include a control module 510 that can cause the automation controls subsystem 410 to control other devices or apparatuses.

In an aspect, the automation control subsystem 410 can be embodied in a thermostat. The biometric monitoring subsystem 130 can determine nearly real-time values of vital signs of subjects in a room. The biometric monitoring subsystem 130 can determine an adjustment to the temperature of the room based on those values, to find a satisfactory (e.g., optimal or nearly optimal) temperature for the room. The biometric monitoring subsystem 130 can then send an instruction message to the automation control subsystem 410, where the instruction message can direct the thermostat to maintain the temperature at about the satisfactory or pre-defined temperature. As another example, the biometric monitoring subsystem 130 can determine vital signs and/or bodily functions indicative of pain. In response to such a determination, the biometric monitoring subsystem 130 can send an instruction to the automation control subsystem 410 to notify one or many of the devices 420 that a pain medication may need to be dispensed, e.g., distributed based on discomfort levels. In an aspect, the device(s) 420 may include a handheld device operated by a nurse or doctor.

In addition, or in yet another example, the biometric monitoring subsystem 130 can determine that one or many subjects are in distress. In response to such a determination, the biometric monitoring subsystem 130 can send an instruction to the automation control subsystem 410 to notify that the subject(s), e.g., first subject 110(1), second subject 110(2), and third subject 110(3) are in distress. The control rule(s) 520 can include parameters that define when alarms and alerts will indicate a patient in distress.

In response to instructions received from the biometric monitoring subsystem 130, the automation control apparatus 410 can cause nearly real-time adjustments to an environment including the monitored subjects, e.g., first subject 110(1), second subject 110(2), and third subject 110(3). For example, adjustments can include changes to volume of ambient sounds; changes to intensity of ambient lighting; changes to light source of ambient lighting; changes to ambient temperature; changes to ambient humidity; a combination thereof or similar. The adjustments can be directed to setting control parameters at or near a satisfactory configuration (e.g., an optimal configuration) in order to achieve a desired state. For example, the biometric monitoring subsystem 130 can determine vital signs and/or bodily functions indicative of a stress level exceeding a threshold or an otherwise undesirable mood and physical condition (e.g., sadness and fatigue). In response to such a determination, the biometric monitoring subsystem 130 can send an instruction to the automation control subsystem 410 to adjust the intensity of ambient lighting and to play soothing sounds or music. In such cases, the device(s) 420 can include multiple light source devices and sounds or music player devices.

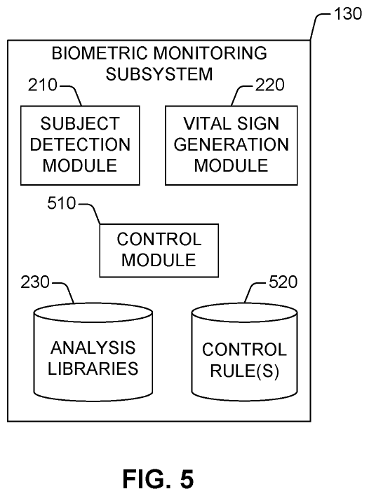

The biometric monitoring subsystem 130 can determine such a desired state based on contactless vital sign monitoring of multiple subjects. In some embodiments, as is illustrated in FIG. 5, the biometric monitoring subsystem 130 can include a control module 510 that can determine the desired state based on values of vital signs corresponding to multiple subjects being monitored.

The biometric monitoring subsystem 130 also can apply one or more control rules to cause automation control subsystem 410 to control one or more devices 420. In one example, a control rule can dictate that an audible alarm be activated when a defined vital sign (e.g., blood pressure or HR) exceeds a first threshold value or is below a second threshold. In such a situation, the biometric monitoring subsystem 130 can direct the automation control subsystem 410 to energize a speaker device to trigger the audible alarm. As is shown in FIG. 5, the control rule(s) can be retained in one or more memory devices 520 (referred to as control rule(s) 520). The control rule(s) 520 can include parameters that can be manually adjusted.

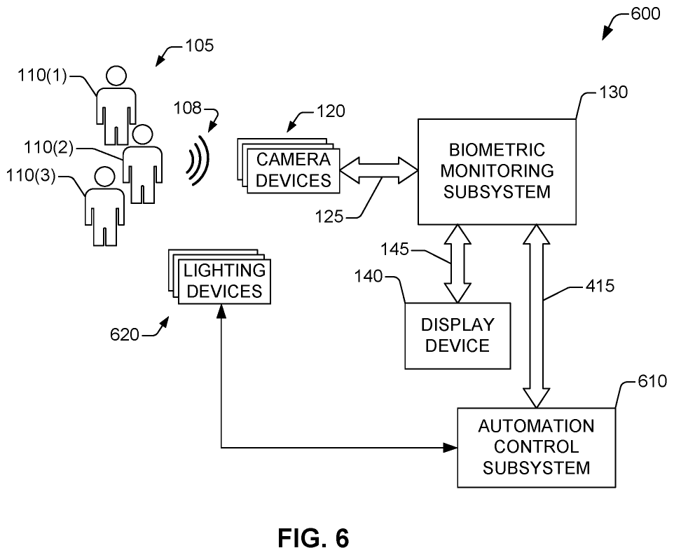

In some situations, the desired state can be an illumination condition of a room housing multiple subjects being monitored are located. The illumination condition can be directed to overcoming technical challenges of the camera devices 120. Thus, as is illustrated in FIG. 6, the biometric monitoring subsystem 130 can cause the automation control subsystem 610 to adjust the operation of lighting devices 620 in an attempt to maintain performance of the determination of values of vital signs. In addition, or in situation involving ambient lighting shifts, the biometric monitoring subsystem 130 can direct one or more of the camera devices 120 to re-acquire bias frames (for calibration) in real-time in order to adjust thresholds of processing algorithms. An example of bias frames includes an initial set of frames that can be used to quantify a baseline/background light levels in the frames. Such bias frames can be used in the real-time processing of images to increase signal-to-noise ratio of the vital sign signals being measured.

In addition, or in some configurations, the control rule(s) 520 can include a criterion that, when fulfilled, can cause the biometric monitoring subsystem 130 to save video signals (visible and/or IR) spanning adjustable time intervals around an event of interest (alarms or alerts) satisfying the criterion.

In some configurations, the system 100 may comprise settings for when patients are no longer visible (i.e., moved off camera). For example, when the subject is out of view or range of the camera device 120, the biometric monitoring subsystem 130 determines to stop monitoring the vital-signs of the subject.

In some configurations, the system 100 comprise settings for when motion is observed that could cause harm (e.g., falling). In response to such a determination, the biometric monitoring subsystem 130 can send an instruction to the automation control subsystem 410 to notify that the subject has fallen. The control rule(s) 520 can include parameters that define when alarms and alerts will indicate a patient is in pain or distress from a fall.

In some configurations, a library of parameter settings is pre-established for medical staff to select when to commence monitoring. For example, fora patient with Sleep Apnea, the patient needs alerts set for when respiratory rates decrease below a certain amount. The control rule(s) 520 can include parameters that define when alarms and alerts will indicate monitoring the patient for sleeping disorder.

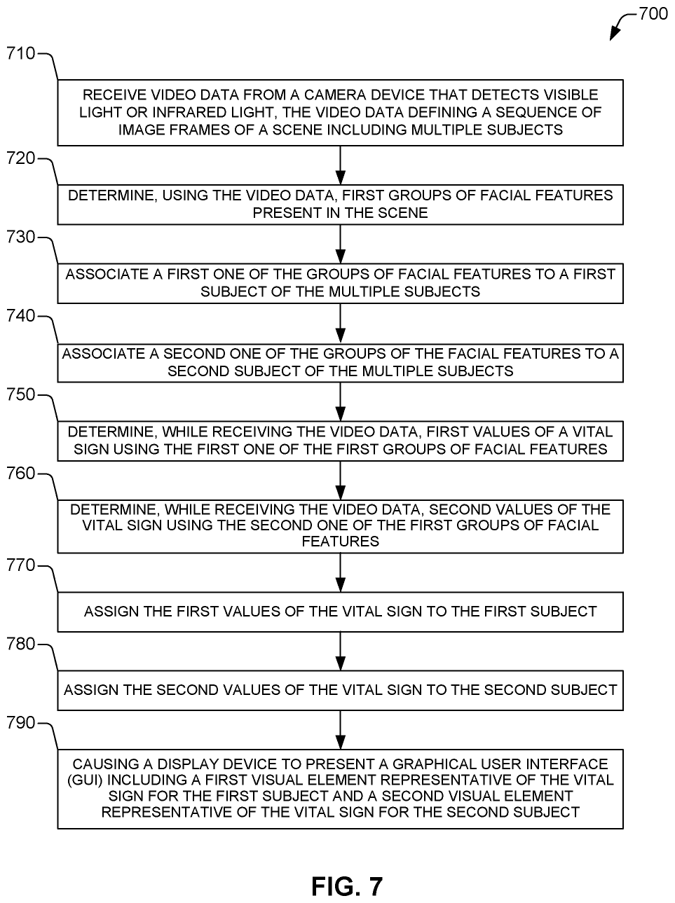

In view of various aspects described herein, example methods can be implemented in accordance with this disclosure. FIG. 7 illustrates an example of method 700 for monitoring vital signs of multiple subjects simultaneously, in nearly real-time. The example method 700 can be performed, entirely or partially, by a computing system (e.g., biometric monitoring system 130). The computing system can include or is functionally coupled to one or many processors, one or many memory devices, other types of computing resources, a combination thereof, or the like. Such processor(s), memory device(s), and computing resources, individually or in a particular combination, permit or otherwise facilitate implementing the example method 700. The computing resources can include operating systems (O/S); software for configuration and/or control of a virtualized environment; firmware; central processing unit(s) (CPU(s)); graphics processing unit(s) (GPU(s)); tensor processing unit(s) (TPU(s)); virtual memory; disk space; interface(s) (I/O interface devices, programming interface(s) (such as application programming interfaces (APIs), etc.); controller devices(s); power supplies; a combination of the foregoing; or the like. Such a computing system can embody, or can include, the biometric monitoring subsystem 130.

In step 710, the computing system can receive video data from a camera device that detects visible light or infrared light. The video data define a sequence of video frames representing respective digital images of a scene including multiple subjects. The respective digital images can be either visible or infrared images. In one example, the multiple subjects can include the first subject 110(1), the second subject 110(2), and the third subject 110(3).

In step 720, the computing system can determine, using the video data, first groups of facial features present in the scene. The facial features can include, for example, faces, noses, foreheads, or a combination of those features.

In step 730, the computing system can associate a first one of the groups of facial features to a first subject of the multiple subjects. For instance, the first subject can be the second subject 110(2). In step 740, the computing system can associate a second one of the groups of facial features to a second subject of the multiple subjects. For instance, the second subject can be the third subject 110(3).

In step 750, the computing system can determine, while receiving the video data, first values of a vital sign (e.g., HR or RR) using the first one of the groups of facial features. In step 760, the computing system can determine, while receiving the video data, first values of the vital sign using the first one of the groups of facial features.

In step 770, the computing system can assign the first values of the vital sign to the first subject 110(1). In step 780, the computing system can assign the second values of the vital sign to the second subject 110(2).

In step 790, the computing system can cause a display device to present a GUI including a first visual element representative of the vital sign for the first subject, and a second visual element representative of the vital sign for the second subject.

Multiple instances of the example method 700 can be implemented in order to generate values for multiple vital signs (e.g., HR, RR, skin temperature, and blood pressure). In an aspect, one instance of the example method 700 can be implemented using visible video signal and another instance can be implemented using infrared video signal. In another aspect, an implementation of the example method 700 can combine together the visible and IR data streams to improve measurements. For example, heart rate can be measured with an infrared camera in a suitable band and a visible camera at the same time. A computing system (e.g., the biometric monitoring subsystem 130) can implement two instances of the example method 700. As a result, two groups of frequency components can be determined from both of those instances independently—one group determined using IR video signal and another group determined using visible video signal. The computing system can then determine respective estimates of HR. In the alternative, the computing system can combine those two groups in order to generate a more accurate estimate of the HR.

In another example, blood pressure can be estimated by monitoring average pixel values on multiple regions of the face in a visible video feed and combining those in a neural network. In some cases, a model defining such a neural network can be retained in the analysis libraries 230 (as shown in FIG. 2). By performing two instances of the example method 700, one using visible video signal and another one using IR video signal, additional data inputs (e.g., IR images or IR image values) to the neural network from the IR regime can improve accuracy of the determined vital signs.

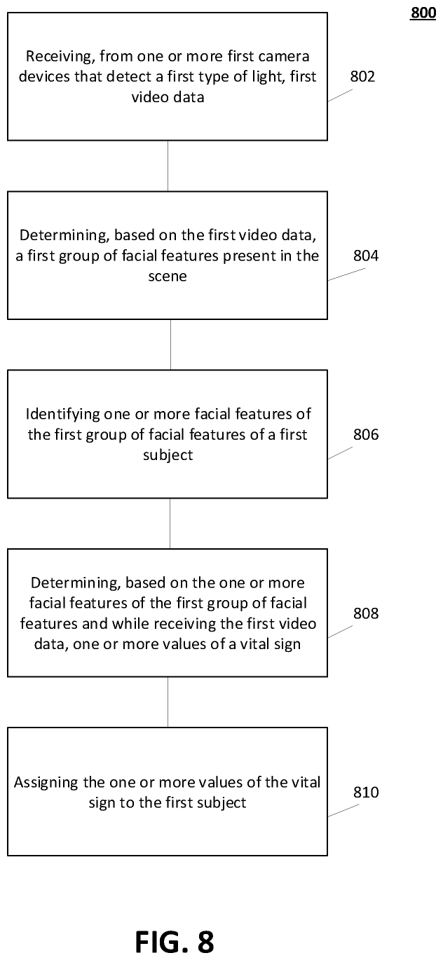

FIG. 8 is a flow chart illustrating an example method 800. At step 802, first video data, from one or more camera device, can be received by a computing device (e.g., biometric monitoring subsystem 130). In an aspect, the first video data can comprise visible video signal defining a sequence of frames, where each frame can represent a digital image formed using visible light. In another aspect, the first video can comprise infrared video signal defining a sequence of frames, where each frame can represent a digital image formed using infrared light. At least one of the camera devices can detect light in the visible portion of the electromagnetic (EM) radiation spectrum (e.g., a range from about 1.63 eV to about 3.26 eV). At least one of the camera devices can detect light in the infrared portion of the EM radiation spectrum (e.g., a range from about 1.24 meV to about 1.63 eV).

At step 804, a first group of facial features present in the image frame can be determined based on the first video data. For example, the first group of facial features can be faces, noses, foreheads, or a combination of facial features. Thus, the group of facial features can be detected in each frame of the sequence of frames included in the received video signal.

In an aspect, the computing device can assign subgroups of the group of facial features to respective subjects present in respective scenes represented by the sequence of frames. More specifically, the computing device can detect a first group of facial features in a frame in the sequence of frames. The computing device can then assign a first subgroup of the first group of facial features with the first subject 110(1), a second subgroup of the first group of facial features to the second subject 110(2), and a third subgroup of the group of facial features to the third subject 110(3). In a consecutive (either successive or subsequent) frame in the sequence of frames, the computing device can detect a second group of facial features. As such, the computing device assigns a first subgroup of the second group of facial features with the first subject 110(1), a second subgroup of the second group of facial features to the second subject 110(2), and a third subgroup of the second group of facial features to the third subject 110(3).

At step 806, one or more of the first group of facial features of the first subject 110(1) can be identified by the computing device. In an aspect, subgroups of the group of facial features to respective subjects present in respective scenes represented by the sequence of frames can be assigned. For example, the assignment can be a first subgroup of the first group of facial features with the first subject 110(1), a second subgroup of the first group of facial features to the second subject 110(2), and a third subgroup of the group of facial features to the third subject 110(3). In a consecutive (either successive or subsequent) frame in the sequence of frames, the computing device can detect a second group of facial features. Accordingly, the assignment can be a first subgroup of the second group of facial features with the first subject 110(1), a second subgroup of the second group of facial features to the second subject 110(2), and a third subgroup of the second group of facial features to the third subject 110(3). In an aspect, the group of facial features can be assigned to the subject and create a record identifying the subject. As such, an association between the group and the subject(s) are generated to obtain identification data pertaining to the subject. In an aspect, the group of facial features can be assigned to the subject anonymously, where an association between the group and the subject is generated, without including data that can identify or can be used to identify the subject. By assigning a subgroup of facial features to a subject in the image corresponding to a frame, the system can determine multiple regions of interest within the image.

At step 808, one or more value of vital sign can be determined based on the one or more of the first group of facial features. In an aspect, the value of vital signs can be analyzed via the multiple regions of interest. As an example, each one of the multiple regions of interest within a frame can include an array of pixels. The image data contained in the array of pixels can be utilized to determine an image value for the regions of interest. In one example, the image value can be determined by evaluating a defined function (e.g., a single-value representing the image content of pixels contained in the regions of interest).

At step 810, the image value of the vital sign can be assigned to the first subject. In an aspect, the image value can be mapped to a temperature value of the skin or the body temperature, or both, of the subject.

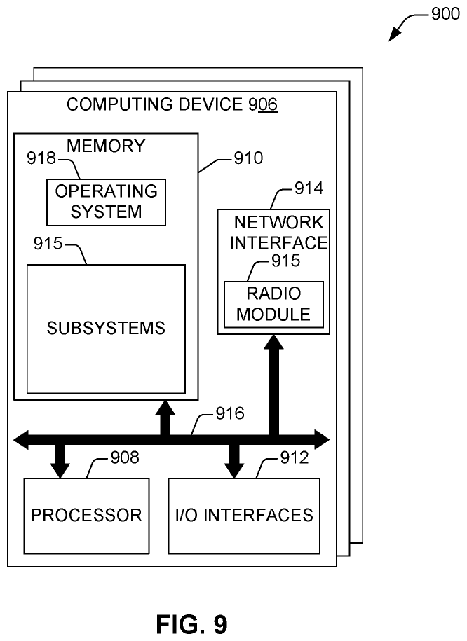

FIG. 9 illustrates an example of a computing environment 900 including examples of a computing device 906. The client device 906 can be a digital computer that, in terms of hardware architecture, can include one or more processor 908 (generically referred to as processor 908), one or more memory devices 910 (generically referred to as memory 910), input/output (I/O) interfaces 912, and network interfaces 914. These components (908, 910, 912, and 914) are communicatively coupled via a communication interface 916. The communication interface 916 can be embodied in or can include, for example, one or more bus architectures or other wireline or wireless connections. One or more of the bus architectures can include an industrial bus architecture, such as an Ethernet-based industrial bus, a controller area network (CAN) bus, a Modbus, other types of fieldbus architectures, or the like. The communication interface 916 can have additional elements, which are omitted for simplicity, such as controller device(s), buffer device(s) (e.g., caches), drivers, repeaters, transmitter device(s), and receiver device(s), to enable communications. Further, the communication interface 916 may include address, control, and/or data connections to enable appropriate communications among the aforementioned components.

The processor 908 can be a hardware device that includes processing circuitry that can execute software, particularly ones stored in the memory 910. In addition, or as an alternative, the processing circuitry can execute defined operations besides those operations defined by software. The processor 908 can be any custom made or commercially available processor, a central processing unit (CPU), a graphical processing unit (GPU), an auxiliary processor among several processors associated with the server device 902 and the client device 906, a semiconductor-based microprocessor (in the form of a microchip or chipset), or generally any device for executing software instructions or performing defined operations. When the server device 902 or the client device 906 is in operation, the processor 908 can be configured to execute software stored within the memory 910, for example, in order to communicate data to and from the memory system 910, and to generally control operations of the server device 902 and the client device 806 according to the software.

The I/O interfaces 912 can be used to receive user input from and/or for providing system output to one or more devices or components. User input can be provided via, for example, a keyboard, a touchscreen display device, a microphone, and/or a mouse. System output can be provided, for example, via the touchscreen display device or another type of display device. I/O interfaces 912 can include, for example, a serial port, a parallel port, a Small Computer System Interface (SCSI), an infrared (IR) interface, a radiofrequency (RF) interface, and/or a universal serial bus (USB) interface.

The network interface 914 can be used to transmit and receive data, metadata, and/or signaling from an external server device, an external client device 806, and other types of external apparatuses over one or more of the network(s) (not depicted in FIG. 8). The network interface 914 also permits transmitting data, metadata, an/or signaling to access control apparatus(es) 905 and receiving other data, metadata, and/or signaling from the access control apparatus(es). The network interface 814 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi), or any other suitable network interface device. Accordingly, as is illustrated in FIG. 8, the network interface 914 in the computing device 906 can include a radio module 915. The network interface 914 may include address, control, and/or data connections to enable appropriate communications on the network(s) 904.

The memory 910 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory 910 may incorporate electronic, magnetic, optical, and/or other types of storage media. In some embodiments, the memory 910 can have a distributed architecture, where various storage devices are situated remotely from one another, but can be accessed by the processor 908.

Software that is retained in the memory 910 may include one or more software components, each of which can include an ordered listing of executable instructions for implementing logical functions. In the example of FIG. 9, the software in the memory 910 of the computing device 906 can include one or more of the subsystems 915 and an operating system (O/S) 918. In some embodiments, the subsystems 915 can include the biometric monitoring subsystem 130.

The O/S 918 essentially controls the execution of other computer programs, such as the O/S 818, and provides scheduling, input-output control, file and data management, memory management, and communication control and related services.

For purposes of illustration, application programs and other executable program components such as the O/S 918 are illustrated herein as discrete blocks, although it is recognized that such programs and components can reside at various times in different storage components of the server device 902 and/or the client device 906. An implementation of the subsystems 915 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

Other example embodiments for contactless vital-sign monitoring of multiple subjects in nearly real-time, in accordance with aspects of this disclosure, include the following:

In some embodiments, the biometric monitoring subsystem 130 can use cameras at different vantage points in order make sure the regions of interest stay on camera at all times.

In addition, or in other embodiments, the biometric monitoring subsystem 130 can have settings to use natural light or a secondary light source.

In addition, or in other embodiments, the camera devices 120 can be on a mobile cart or fixed in a room.

In addition, or in other embodiments, the biometric monitoring subsystem 130 can include steering cameras that can move to better align and track patients.

In addition, or in other embodiments, the biometric monitoring subsystem 130 can use cameras synchronized or unsynchronized frames.

In addition, or in other embodiments, one or many of the monitoring environments described herein (FIG. 1, FIG. 4, and/or FIG. 6) can have an option to be battery powered.

In addition, or in other embodiments, one or many microphones can be functionally coupled to the biometric monitoring subsystem 130 that can provide audio signals in addition to the video signals generated and provided by the camera devices 120.

In addition, or in other embodiments, the biometric monitoring subsystem 130 can include options for not recording into memory certain data types (e.g., video) in order to accommodate patient specific views on being recorded.

In addition, or in other embodiments, the cameras devices 120 can include auto-focus capabilities provided by the camera manufacturers.

In addition, or in other embodiments, video signals can be collected in the presence or absence of a backdrop or another type of a background. In case a backdrop is present, reference objects of known geometries can be placed in the background of camera device(s) video signals in order to assist in tracking and/or distance of subjects to a camera device.

In addition, or in other embodiments, fast steering mirrors can be included in the monitoring environments described herein (FIG. 1, FIG. 4, and/or FIG. 6) to deliver better tracking and resolution.

In addition, or in other embodiments, the biometric monitoring subsystem 130 can use dithering to improve resolution.

In addition, or in other embodiments, the biometric monitoring subsystem 130 can include a performance validation of algorithms using baseline measurements from commercially available vitals monitoring systems.

In addition, or in other embodiments, constant thermal reference in FOV of respective at least one of the camera devices 120 can be included in the monitoring environments described herein (FIG. 1, FIG. 4, and/or FIG. 6).

In addition, or in other embodiments, constant red, green, or blue reference in the FOV of respective at least one of the camera devices 120 can be included in the monitoring environments described herein (FIG. 1, FIG. 4, and/or FIG. 6).

In addition, or in other embodiments, oscillating thermal reference in the FOV of respective at least one of the camera devices 120 can be included in the monitoring environments described herein (FIG. 1, FIG. 4, and/or FIG. 6).

In addition, or in other embodiments, oscillating red, green, and/or blue reference in the FOV of respective at least one of the camera devices 120 can be included in the monitoring environments described herein (FIG. 1, FIG. 4, and/or FIG. 6).

In addition, or in other embodiments, the monitoring environments described herein (FIG. 1, FIG. 4, and/or FIG. 6) can have an option that provides operators and patients with optimal camera placement or body positioning on camera.

In addition, or in other embodiments, the biometric monitoring subsystem 130 can identify other items in the room from a library of medical devices (e.g., IV fluid solution bags).

In addition, or in other embodiments, the monitoring environments described herein (FIG. FIG. 1, FIG. 4, and/or FIG. 6) can use or otherwise leverage patterned clothing on the subjects in order to assist with tracking.

In addition, or in other embodiments, the monitoring environments described herein (FIG. 1, FIG. 4, and/or FIG. 6) can include options for manually selecting regions of interest for tracking.

In addition, or in other embodiments, the monitoring environments described herein (FIG. 1, FIG. 4, and/or FIG. 6) can include options for manually selecting areas of the camera feed that do not have tracking capabilities (e.g., those pixels are not examined by the algorithms).

In the event of identical twins, in some embodiments, the monitoring environments described herein (FIG. 1, FIG. 4, and/or FIG. 6) can leverage manual tags or clothing to separate individuals and their vitals.

In the event of identical twins, in some embodiments, the monitoring environments described herein (FIG. 1, FIG. 4, and/or FIG. 6) can include a manual option for selecting areas of interest in the frame to report average pixel values as a function of time. This is useful for calibrating the systems included in those environments.

In addition, or in other embodiments, the biometric monitoring subsystem 130 can leverage historical time series data from a subject to enable more accurate calculations of present vitals. Such data can be retained in a data repository functionally coupled to the biometric monitoring subsystem 130.

In addition, or in other embodiments, the biometric monitoring subsystem 130 can leverage multiple colors of a visible video signal in order to improve signal-to-noise ratio and/or accuracy of determinations of vital signs (e.g., both blue and red pixels for heart rate).

In addition, or in other embodiments, the biometric monitoring subsystem 130 can leverage data from multiple, separate areas of the body to assist with vitals calculations (e.g., hands and face or different parts of the face).

In addition, or in other embodiments, the biometric monitoring subsystem 130 can use error correction algorithms to correct faulty data.

In addition, or in other embodiments, the biometric monitoring subsystem 130 can use the audio of the patient and room to improve vitals calculations.

In addition, or in other embodiments, the monitoring environments described herein (FIG. 1, FIG. 4, and/or FIG. 6) can include a nighttime setting that changes to algorithm parameters to accommodate low-light conditions.

In addition, or in other embodiments, the monitoring environments described herein (FIG. 1, FIG. 4, and/or FIG. 6) can include an option to be linked with vitals monitoring from more conventional systems. The data from conventional vitals monitoring can be incorporated into the data from the video feeds to improve accuracy.

In addition, or in other embodiments, the monitoring environments described herein (FIG. 1, FIG. 4, and/or FIG. 6) can include settings to save algorithm parameters for individual patients and recall those settings at a later time.

In addition, or in other embodiments, the monitoring environments described herein (FIG. 1, FIG. 4, and/or FIG. 6) can use motion of the body or clothing to compute vitals. An example for this would be that, if the patient is intubated, the system can track chest movements to compute respiratory rate.

In addition, or in other embodiments, the monitoring environments described herein (FIG. 1, FIG. 4, and/or FIG. 6) can include a library of algorithm parameters for various patient types (male, female, adult, child, etc.). Such a library can be retained in the analysis library FIG. 1, FIG. 4, and/or FIG. 6, for example.

In addition, or in other embodiments, the monitoring environments described herein (FIG. 1, FIG. 4, and/or FIG. 6) can include options for both manual and automatic tuning of algorithm parameters in real time.

In addition, or in other embodiments, the monitoring environments described herein (FIG. 1, FIG. 4, and/or FIG. 6) can include settings for selecting different vitals of interest in real time.

In addition, or in other embodiments, the monitoring environments described herein (FIG. 1, FIG. 4, and/or FIG. 6) can be trained to detect a sudden loss of consciousness and sound alerts/alarms.

In addition, or in other embodiments, the biometric monitoring subsystem 130 can retain data defining vital signs, samples of video, and raw pixel data. At least some of that information can be downloaded and/or linked with electronic health records. Data can be accessible remotely and can be stored in the cloud.

Systems and methods can be applied in numerous scenarios:

Contactless Monitoring. Monitoring body temperature, airways, and blood oxygenation/circulation can be critical components of medical diagnoses and treatment. Although emergency rooms and hospitals are outfitted with vitals monitoring capabilities, there are many types or patients that would benefit from contactless vitals monitoring capability. These can include, among others:

Neo-natal infants who have sensitive skin,
Burn victims,
Patients with autism that are hyper-sensitive to skin contact or other physical simulation,
Patients with sleep disorders.
Patients with highly infectious diseases, where exposure can be limited using vitals monitoring and assessment through windows of quarantined rooms.
Dangerous patients in prisons.
In addition, having vitals monitoring available from live camera streams would allow hospital to begin care and triaging the moment someone enters a waiting room. Lastly, contactless monitoring of patient could also be tailored to work in enclosed diagnostic scanners such as MRIs.

Crowd-Based Monitoring. Monitoring health vitals in other crowded areas, such as assisted living facilities, schools, and/or airports, can permit early detection of potential outbreaks of influenza and/or other types of coronavirus outbreaks. In addition, vitals monitoring in a crowd can may also be used to detect those that are nervous and may have malicious intent.

Telemedicine. The telemedicine industry is also growing rapidly, and with the recently affordable camera attachments for computing devices, e.g., personal computers, portable computers, smart phones, mobile phones, and others, advancements in video health monitoring will inevitably improve patient outcomes for patients who live far hospitals and require frequent assessment.

Military Medicine. Smart thermal/visible cameras can be applied to monitoring vital sign of healthy, moving soldiers. Such monitoring can provide predictions about troops health/performance, including heat exhaustion, physical fatigue, alertness-fitness duty, and also can provide triage/diagnosis information. Not only can the systems and methods improve conventional wearable vital monitoring devices, but the systems and methods can operate in scenarios where wearable devices are not ideal or cannot operate efficiently. Those scenarios can include, for example, monitoring of multiple injured soldiers in battlefield or prolonged field care settings. Remote monitoring of health/performance can permit commanders or medical officers to manage multiple soldiers simultaneously for fitness, training resiliency, and/or injury prevention.

There are other scenarios, such as Veteran Affairs (VA) Medical Centers and large metropolitan hospitals, where high patient volume can create significant challenges for the hospital and the waiting rooms in the hospital. The systems and methods can be used for rapid triage in crowded rooms. As a result, in sharp contrast to conventional technologies, the systems and methods can significantly reduce the burden placed on Emergency Department doctors, nurses, and staff.

Contactless Fatigue Monitoring. Fatigue monitoring has already been studied in various critical areas of our lives. For example, within the medical community, trained radiologists must review vast amounts of patient x-rays that require the human eye and the decision-making capabilities of the human brain; the interpretation of x-rays requires focused experts to sort through large amounts of data and produce actionable intelligence that can save lives. Radiologist fatigue and resulting errors have been studied in the literature along with metrics that are visible to the human eye (e.g. eyestrain tests, reaction time, break frequency, sleep patterns, etc.).

The technologies disclosed herein also can provide fatigue monitoring of subjects, such as military personnel. In one example, the systems and methods can provide contactless remote cardiopulmonary sensing. In addition, for subjects engages in detail-intensive activities, the systems and methods can provide nearly real-time monitoring the cognitive loads of those subjects, who can review data feeds provided by an abundance of sensors used for intelligence gathering, for example.

Vitals Monitoring for Animals in Captivity (Shelters, Farms, Research Studies, Zoos, etc.). The systems and methods also can provide real-time monitoring of vital signs of multiple animals in captivity. Contactless monitoring with nearly real-time updates can identify animals in distress or otherwise in need of attention. In one example scenario, the systems and methods can be applied to monitoring large populations of cattle. Monitoring vital signs of multiple animals simultaneously can mitigate risk and reduce cost of mouse model animal studies.

Sports Medicine—The systems and methods for contactless vital sign monitoring in nearly real-time can be applied to tracking athletic performance and fatigue. Heart rate, respiratory rate, and bodily temperatures are all key indicators of physical fitness, and can be used to support cardiovascular and weight training and/or physical therapy. Multi-subject, simultaneous contactless monitoring in nearly real-time of team sports that do not use padding (soccer, volleyball, swimming, etc.) can provide real-time monitoring of vital signs that can serve as a diagnostic tool that coaches and/or managers can utilized in real-time. Such monitoring can be use to device gameplay and other game strategies.

Baby Monitors. —The systems and methods also can improve conventional baby monitors. The systems and methods can be provide baby monitors without wearable devices. In some cases, the systems and methods can be combined with traditional baby monitors to provide contactless vital signs monitoring in nearly real-time and report on breathing issues, heart rate irregularities, or fever, for example.

While the technologies (e.g., techniques, computer program products, devices, and systems) of this disclosure have been described in connection with various embodiments and specific examples, it is not intended that the scope be limited to the particular embodiments put forth, as the embodiments herein are intended in all respects to be illustrative rather than restrictive

As used in this application, the terms “environment,” “system,” “module,” “component,” “architecture,” “interface,” “unit,” and the like are intended to encompass an entity that includes either hardware, software, or a combination of hardware and software. Such an entity can be embodied in, or can include, for example, a signal processing device. In another example, the entity can be embodied in, or can include, an apparatus with a defined functionality provided by optical parts, mechanical parts, and/or electronic circuitry. The terms “environment,” “system,” “engine,” “module,” “component,” “architecture,” “interface,” and “unit” can be utilized interchangeably and can be generically referred to functional elements.

A component can be localized on one processing device or distributed between two or more processing devices. Components can communicate via local and/or remote architectures in accordance, for example, with a signal (either analogic or digital) having one or more data packets (e.g., data from one component interacting with another component in a local processing device, distributed processing devices, and/or across a network with other systems via the signal).

As yet another example, a component can be embodied in or can include an apparatus with a defined functionality provided by mechanical parts operated by electric or electronic circuitry that is controlled by a software application or firmware application executed by a processing device. Such a processing device can be internal or external to the apparatus and can execute at least part of the software or firmware application. Still in another example, a component can be embodied in or can include an apparatus that provides defined functionality through electronic components without mechanical parts. The electronic components can include signal processing devices to execute software or firmware that permits or otherwise facilitates, at least in part, the functionality of the electronic components. For the sake of illustration, an example of such processing device(s) includes an integrated circuit (IC), an application-specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed or otherwise configured (e.g., manufactured) to perform the functions described herein.

In some embodiments, components can communicate via local and/or remote processes in accordance, for example, with a signal (either analog or digital) having one or more data packets (e.g., data from one component interacting with another component in a local system, distributed system, and/or across a network such as a wide area network with other systems via the signal). In addition, or in other embodiments, components can communicate or otherwise be coupled via thermal, mechanical, electrical, and/or electromechanical coupling mechanisms (such as conduits, connectors, combinations thereof, or the like). An interface can include input/output (I/O) components as well as associated processors, applications, and/or other programming components.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language generally is not intended to imply that features, elements, and/or operations are in any way required for one or more implementations or that one or more implementations necessarily include logic for deciding, with or without user input or prompting, whether these features, elements, and/or operations are included or are to be performed in any particular implementation.

The flowchart and block diagrams in the Figures illustrate the architecture, functionality, and operation of possible implementations of examples of systems, methods, and computer program products according to various embodiments of the present disclosure. In this regard, each block in the flowchart or block diagrams may represent a module, segment, or portion of instructions, which includes one or more machine-executable or computer-executable instructions for implementing the specified operations. It is noted that each block of the block diagrams and/or flowchart illustration, and combinations of blocks in the block diagrams and/or flowchart illustration, can be implemented by special purpose hardware-based devices that perform the specified functions or operations or carry out combinations of special purpose hardware and computer instructions.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

What has been described herein in the present specification and annexed drawings includes examples of systems, apparatuses, devices, and techniques for simultaneous monitoring of vital signs of multiple subjects in real time. It is, of course, not possible to describe every conceivable combination of components and/or methods for purposes of describing the various elements of the disclosure, but it can be recognized that many further combinations and permutations of the disclosed elements are possible. Accordingly, it may be apparent that various modifications can be made to the disclosure without departing from the scope or spirit thereof. In addition, or as an alternative, other embodiments of the disclosure may be apparent from consideration of the specification and annexed drawings, and practice of the disclosure as presented herein. It is intended that the examples put forth in the specification and annexed drawings be considered, in all respects, as illustrative and not limiting. Although specific terms are employed herein, they are used in a generic and descriptive sense only and not for purposes of limitation.

What is claimed is:

1. A method, comprising:
receiving, from one or more first camera devices that detect a first type of light, first video data, the first video data defining a first sequence of image frames of a scene including a plurality of subjects;
determining, based on the first video data, a first group of facial features present in the scene;
identifying one or more facial features of the first group of facial features of a first subject of the plurality of subjects;
determining, based on the identified one or more facial features of the first group of facial features and while receiving the first video data, one or more values of a first vital sign; and
assigning the one or more values of the first vital sign to the first subject.

2. The method of claim 1, further comprising:
receiving, from one or more second camera devices that detect a second type of light, second video data, the second video data defining a second sequence of image frames of the scene including the plurality of subjects;
determining, based on the second video data, a second group of facial features present in the scene;
identifying one or more facial features of the second group of facial features of a second subject of the plurality of subjects;
determining, based on the identified one or more facial features of the second group of facial features and while receiving the second video data, one or more values of a second vital sign; and
assigning the one or more values of the second vital sign to the second subject.

3. The method of claim 1, further comprising:
identifying one or more facial features of a third group of facial features of a third subject of the plurality of subjects;
determining, based on the identified one or more facial features of the third group of facial features, one or more values of a third vital sign; and
assigning the one or more values of the third vital sign to the third subject.

4. The method of claim 2, wherein the first type of light is visible light and the second type of light is infrared light.

5. The method of claim 2, further comprising causing a display device to present a graphical user interface (GUI) including a first visual element representative of the first vital sign and a second visual element representative of the second vital sign.

6. The method of claim 2, further comprising performing an operation based at least on the one or more values of the first vital sign or the one or more values of the second vital sign.

7. The method of claim 2, wherein the second vital sign is skin temperature, and wherein the determining, based on the identified one or more facial features of the second group of facial features and while receiving the second video data, the one or more values of the second vital sign using the one or more facial features of the second group of facial features comprises,
identifying particular facial features in the one or more facial features of the second group of facial features;
determining a region containing the particular facial features within a digital image corresponding to an image frame of the second sequence of image frames;
determining an image value representative of an average pixel intensity within the region; and
mapping the image value to a temperature value of the skin temperature.

8. The method of claim 1, wherein a visible image comprises the first sequence of image frames, and wherein the determining, based on the first video data, the first group of facial features present in the image frame comprises,
generating a time series of image values corresponding to the first sequence of image frames, each image value in the time series identifying an average of pixel values within a defined region in a respective image frame in the sequence;
generating multiple frequencies by applying a fast Fourier transform to the time series;
selecting a first frequency of the multiple frequencies, the first frequency satisfying a magnitude criterion; and
generating the one or more values of the first vital sign using the first frequency.

9. The method of claim 1, further comprising
obtaining data defining an exempt subject excluded from a determination of the first vital sign;
determining that a particular group of the first group of facial features corresponds to the exempt subject; and
excluding the particular group from an image analysis to determine the first vital sign.

10. An apparatus comprising:
at least one processor; and
at least one memory device having executable instructions encoded thereon that, in response to execution, cause a computing device to perform or facilitate operations comprising:
receive, from one or more first camera devices that detect a first type of light, first video data, the first video data defining a first sequence of image frames of a scene including a plurality of subjects;
determine, based on the first video data, a first group of facial features present in the scene;
identify one or more facial features of the first group of facial features of a first subject of the plurality of subjects;
determine, based on the identified one or more facial features of the first group of facial features and while receiving the first video data, one or more values of a first vital sign; and
assign the one or more values of the first vital sign to the first subject.

11. The apparatus of claim 10, wherein the processor executable instructions, when executed by the one or more processors, further cause the computing device to:
receive, from the one or more camera devices that detect a second type of light, second video data, the second video data defining a second sequence of image frames of the scene including the plurality of subjects;
determine, based on the second video data, a second group of facial features present in the image frames;
identify one or more facial features of the second group of facial features of a second subject of the plurality of subjects;
determine, based on the identified facial features one or more of the second group of facial features and while receiving the second video data, one or more values of a second vital sign; and
assign the one or more values of the second vital sign to the second subject.

12. The apparatus of claim 11, wherein the processor executable instructions, when executed by the one or more processors, further cause the computing device to:
identify one or more facial features of a third group of facial features of a third subject of the plurality of subjects;
determine, based on the identified one or more facial features of the third group of facial features, one or more values of a third vital sign; and
assign the one or more values of the third vital sign to the third subject.

13. The apparatus of claim 11, wherein the first type of light is visible light and the second type of light is infrared light.

14. The apparatus of claim 11, wherein the processor executable instructions, when executed by the one or more processors, further cause the computing device to:
display a graphical user interface (GUI) including a first visual element representative of the first vital sign and a second visual element representative of the second vital sign.

15. The apparatus of claim 11, wherein the processor executable instructions, when executed by the one or more processors, further cause the computing device to:
cause the apparatus to perform an operation based at least on the one or more values of the first vital sign or the one or more values of the second vital sign.

16. The apparatus of claim 11, wherein the processor executable instructions, when executed by the one or more processors, further cause the computing device to:
identify particular facial features in the one or more facial features of the second group of facial features;
determine a region containing the particular facial features within a digital image corresponding to the image frame of the second sequence of image frames;
determine an image value representative of an average pixel intensity within the region; and
map the image value to a temperature value of a skin temperature.

17. The apparatus of claim 11, wherein the processor executable instructions, when executed by the one or more processors, further cause the computing device to:
generate a time series of image values corresponding to the sequence of image frames, each image value in the time series identifying an average of pixel values within a defined region in a respective image frame in the sequence;
generate multiple frequencies by applying a fast Fourier transform to the time series;
select a first frequency of the multiple frequencies, the first frequency satisfying a magnitude criterion; and
generate the one or more values of the first vital sign using the first frequency.

18. The apparatus of claim 11, wherein the processor executable instructions,
when executed by the one or more processors, further cause the computing device to:
obtain data defining an exempt subject excluded from a determination of a vital sign;
determine that a particular group of the groups of facial features corresponds to the exempt subject; and
exclude the particular group from an image analysis to determine the vital sign.

19. A system, comprising:
a plurality of camera devices comprising a first camera device that detects a first type of light and a second camera device that detects a second type of light; and
a computing device in communication with the plurality of camera devices, wherein the computing device is configured to:
receive, from the first camera device, first video data, the first video data defining a first sequence of image frames of a scene including a plurality of subjects;
determine, based on the first video data, a first group of facial features present in the scene;
identify one or more facial features of the first group of facial features of a first subject of the plurality of subjects;
determine, based on the identified one or more facial features of the first group of facial features and while receiving the first video data, one or more values of a first vital sign; and
assign the one or more values of the first vital sign to the first subject.

20. The system of claim 19, wherein the computing device is configured to:
receive, from the second camera device, second video data, the second video data defining a second sequence of image frames of the scene including the plurality of subjects;
determine, based on the second video data, a second group of facial features present in the scene;
identify one or more facial features of the second group of facial features of a second subject of the plurality of subjects;
determine, based on the identified one or more facial features of the second group of facial features and while receiving the second video data, one or more values of a second vital sign; and
assign the one or more values of the second vital sign to the second subject.
